(19) United States
(12) Patent Application Publication
(10) Pub. No.: US 2021/0369261 A1
(43) Pub. Date: Dec. 2, 2021

(54) SUTURE ANCHOR INSERTER

(71) Applicant: Flower Orthopedics Corporation, Horsham, PA (US)

(72) Inventors: Angelo PATRINICOLA, Audubon, NJ (US); Michael SIMONSON, Hatboro, PA (US); Kevin RZASA, Wilmington, DE (US)

(21) Appl. No.: 17/286,728

(22) PCT Filed: Oct. 18, 2019
(86) PCT No.: PCT/US2019/056889

§ 371 Date: Apr. 19, 2021

Related U.S. Application Data
(60) Provisional application No. 62/747,836, filed on Oct. 19, 2018.

(51) Int. Cl. A61B 17/04; A61B 17/06; A61B 17/16; A61B 90/30

(52) U.S. Cl. CPC A61B 17/0401; A61B 17/06061; A61B 2017/0409; A61B 90/30; A61B 17/1615

(57) ABSTRACT

The present invention provides improved suture inserter devices. The inserter devices are configured to securely hold a suture anchor with elements for routing and securing an attached suture thread and suture needle.

### CROSS-REFERENCE TO RELATED APPLICATIONS

This application claims priority to U.S. Provisional Patent Application No. 62/747,836, filed Oct. 19, 2018, the contents of which are incorporated by reference herein in its entirety.

### BACKGROUND OF THE INVENTION

Suture anchors are commonly used to treat tendon and ligament ruptures and to fix soft tissue to bone. The suture anchor construct typically consists of a suture with two attached needles, which is affixed to the anchor body. These suture anchor constructs are usually attached to an inserter device, which interfaces with the anchor and also provides a way of keeping the suture secured. Providing a suture anchor inserter with a suture anchor having a thread and an attached suture needle would enhance efficiency in surgical procedures but requires safe management of the exposed needle.

There is a need in the art for an improved suture anchor inserter device that safely secures a suture thread and suture needle. The present invention meets this need.

### SUMMARY OF THE INVENTION

In one aspect, the present invention relates to a suture anchor inserter device comprising: a substantially cylindrical handle positioned at a proximal end, the handle having an outer surface comprising at least one embedded groove circumscribing the handle, a planar surface adjacent to the at least one embedded groove, and a plurality of posts attached to the planar surface; an elongate, substantially cylindrical shank extending from the handle towards a distal end, the shank having an outer surface, a lumen at least partially throughout, a window on the outer surface connected to the lumen, and a driver bit positioned at an open distal tip; and a cap sized to fit over the planar surface of the handle and the at least one embedded groove.

In one embodiment, the plurality of posts are spaced to fit a suture needle wedged in-between. In one embodiment, the at least one embedded groove forms a continuous channel circumscribing the handle. In one embodiment, the handle further comprises a trough embedded in the outer surface, the trough extending from the at least one embedded groove to the shank. In one embodiment, the shank comprises a peg extending from its outer surface, the peg being positioned near handle 16. In one embodiment, the driver bit is selected from the group consisting of: a hex shape, a square shape, a triangle shape, a cross shape, a slot shape, and a Torx shape. In one embodiment, the shank further comprises a trough embedded in its outer surface, the trough extending from the window to the handle. In one embodiment, the cap is attached to the handle by a friction fit, a snap fit, or a threaded fitting. In one embodiment, the planar surface is detachable from the handle along the at least one embedded groove. In one embodiment, the device further comprises a light source connected to a power supply. In one embodiment, the light source is attached to the handle, the shaft, or both. In one embodiment, the device further comprises a drill bit positioned at the proximal end of the handle. In one embodiment, the cap comprises a hole permitting the drill bit to extend through the cap.

In another aspect, the present invention relates to a suture anchor inserter kit, comprising: the suture anchor inserter device of the present invention; at least one suture thread; at least one suture anchor; and at least one suture needle.

In one embodiment, the at least one suture anchor is prethreaded with a length of suture thread. In one embodiment, the length of suture thread is prethreaded onto a suture needle. In one embodiment, the suture anchor is attached to the driver bit of the suture anchor inserter device, such that the suture thread is routed through the lumen of the shank, out of the window of the shank, and wound tautly around the at least one embedded groove of the handle. In one embodiment, the suture anchor inserter device is provided with at least one suture needled wedged in-between the plurality of posts. In one embodiment, the at least one suture needle is wedged in-between the plurality of posts and prethreaded with the length of suture thread wound tautly around the at least one embedded groove of the handle. In one embodiment, the cap is fitted over the planar surface of the handle and the at least one embedded groove and covers the length of suture thread wound around the at least one embedded groove and the at least one suture needle wedged in-between the plurality of posts.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1A and FIG. 1B depict an exemplary inserter device. FIG. 1A depicts a side view of an exemplary inserter device. FIG. 1B depicts a magnified view of the proximal storage end of the inserter device.

FIG. 2A and FIG. 2B depict an exemplary inserter device having a suture anchor and thread with a cover protecting a suture needle. FIG. 2A is a side view. FIG. 2B is a top view.

### DETAILED DESCRIPTION

The present invention provides improved suture inserter devices. The inserter devices are configured to securely hold a suture anchor with elements for routing and securing an attached suture thread and suture needle.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Suture Anchor Inserter Device

The present invention provides inserter devices for suture anchors. The inserter devices include channels and pegs for routing and securing suture threads and suture needles that are attached to a suture anchor. The inserter devices include removable caps that cover the suture needle until needed.

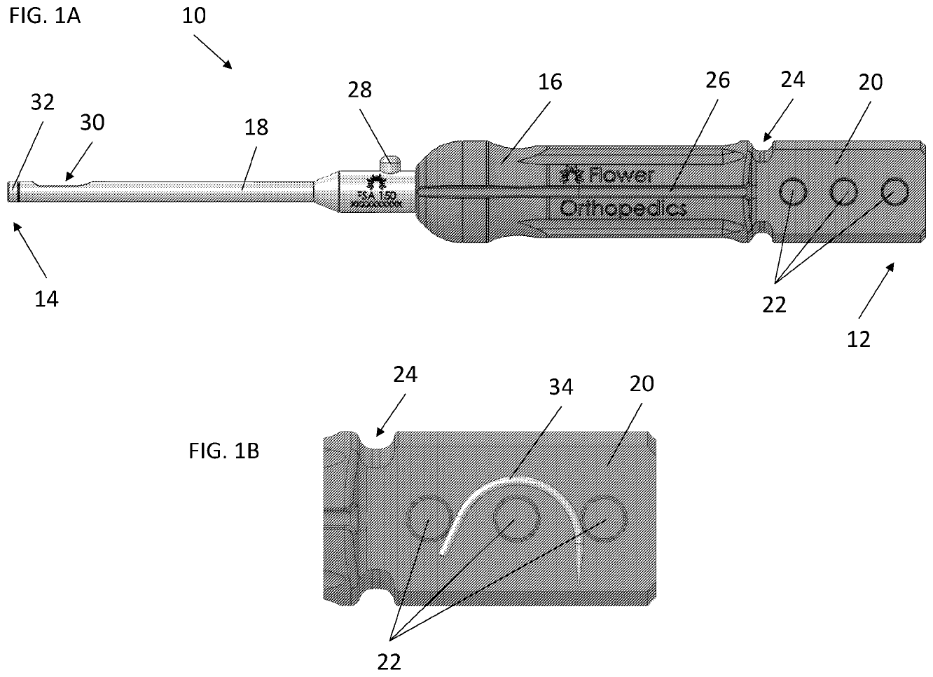

Referring now to FIG. 1A and FIG. 1B, an exemplary inserter device 10 is depicted. Device 10 comprises a handle 16 at a proximal end 12 connected to an elongate shank 18 at a distal end 14. Handle 16 has a substantially cylindrical shape and can include one or more textured features on an outer surface to enhance grip, including but not limited to ridges, ribbing, indents, and the like. Handle 16 comprises a storage end 20 having a planar surface with a plurality of posts 22 attached to the planar surface. As shown in FIG. 1B, the plurality of posts 22 are spaced apart such that a needle 34 can be wedged and secured between the plurality of posts 22. While device 10 is depicted having three posts 22 in a linear arrangement, it should be understood that device 10 is not limited in the number of posts 22 and in the arrangement of posts 22. For example, storage end 20 can have a planar surface with a grid of posts 22 spaced apart to fit one or more curved needles 34 as well as straight needles. Handle 16 comprises at least one groove 24 distal to storage end 20, each groove 24 forming segments of an indented channel circumscribing the outer surface of handle 16. In some embodiments, the at least one groove 24 can be connected to each other to form a continuous groove around the outer surface of handle 16. The at least one groove 24 provides a depression sized to secure a length of suture thread that is wound around handle 16. Handle 16 further comprises trough 26 embedded within its outer surface. Trough 26 extends from the at least one groove 24 to the distal end of handle 16 and has a depth sized to fit at least one suture thread.

Shank 18 extends from the distal end of handle 16 and has a substantially cylindrical shape with a smaller diameter than handle 16 to facilitate entry into narrow spaces. In some embodiments, shank 18 is hollow, having a lumen therethrough. In other embodiments, shank 18 is at least partially hollow, having a lumen extending from distal end 14 to a position along shank 18. Shank 18 comprises aperture 30 and driver bit 32. Driver bit 32 is positioned at the distal end of shank 18 and is a shaped opening to the lumen of shank 18 that is adapted to fit and drive a suture anchor. Driver bit 32 thereby can have any suitable construction that is commonly used to drive suture anchors, including but not limited to a hex shape, a square shape, a triangle shape, a cross shape, a slot shape, a Torx shape, and the like. Aperture 30 is positioned on an outer surface of shank 18 and is open to the lumen of shank 18. In some embodiments, shank 18 further comprises peg 28 extending from its outer surface positioned near handle 16. In some embodiments, shank 18 further comprises a trough embedded within its outer surface extending from aperture 30 to a peg 28 or the proximal end of shank 18, wherein the trough can be similar to trough 26 or is a continuation of trough 26 that has a depth sized to fit at least one suture thread.

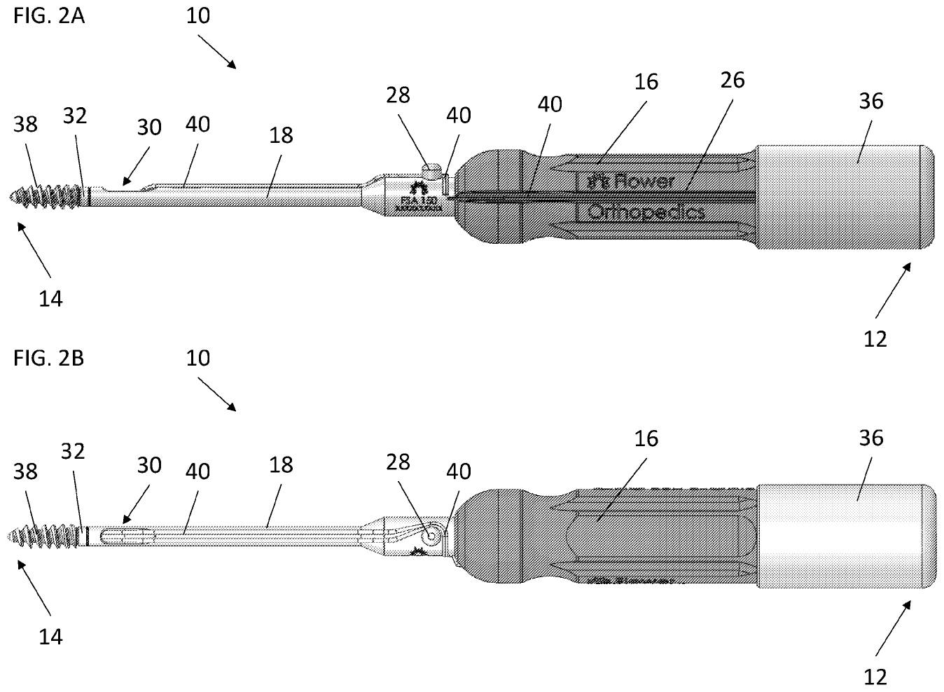

Referring now to FIG. 2A and FIG. 2B, an exemplary inserter device 10 is depicted with a suture anchor 38 and cap 36. Suture anchor 38 with an attached suture thread 40 is inserted and secured into driver bit 32. Suture thread 40 exits the lumen of shank 18 by way of aperture 30 and is routed through trough 26. While not visible in FIG. 2A and FIG. 2B, the remaining length of suture thread 40 is wound around the at least one groove 24. In the depicted embodiment, peg 28 is provided near handle 16 and can be used to wrap a portion of suture thread 40. Peg 28 can thereby be used to maintain the tautness of suture thread 40 and to hold suture thread 40 close to the outer surface of shank 18.

Cap 36 fits over storage end 20 and the at least one groove 24 and provides a protective and sterile cover for a length of suture thread 40 wound around the at least one groove 24 as well as one or more suture needles 34 secured to storage end 20. Cap 36 can be attached by any suitable means commonly used in the art, including a friction-fit, a snap fit (such as by way of a tab on cap 36 that fits into the at least one groove 24), a threaded fitting (such as by way of a threaded outer region on storage end 20 and a threaded inner region of cap 36), and the like.

The components of the inserter devices of present invention can be modified in any suitable manner to enhance their function. For example, in some embodiments, device 10 can include one or more light sources and portable power supplies attached to handle 16 or shank 18. Suitable light sources include LEDs and optical fiber ends connectable to an exterior light, whereupon the light sources can illuminate a site of suture anchor insertion during use. In another example, device 10 can include a drill bit attached at proximal end 12, wherein the drill bit is compatible with powered drills commonly used in the art. The drill bit enables the entire device 10 to be driven with a powered drill for ease of suture anchor insertion. The drill bit can be exposed using a cap 36 having a hole at its proximal end, such that the cap 36 can cover grooves 24 and storage end 20 without covering the drill bit. In another example, storage end 20 can be detachable from handle 16. A detachable storage end 20 can be initially attached by way of a friction fit, a snap fit, a threaded fitting, or by a breakaway mechanism, and can be removed from handle 16 along the indent of the at least one groove 24 to quickly unwind a length of suture thread 40 from device 10.

The inserter devices of the present invention can be constructed using any suitable method known in the art. The methods may vary depending on the materials used. For example, certain components can substantially comprise a plastic or polymer that may be milled from a large block or injection molded. Likewise, certain components can substantially comprise a metal that may be milled, cast, etched, or deposited by techniques such as chemical vapor deposition, spraying, sputtering, and ion plating. In some embodiments, the devices may be made using 3D printing techniques commonly used in the art.

The present invention also includes kits comprising the inserter devices described elsewhere herein. The kits may provide one or more inserter devices with a length of suture thread, one or more suture anchors, and one or more suture needles. In some embodiments, the one or more suture anchors can be prethreaded with a length of suture thread. In some embodiments, the kits may provide one or more inserter devices with a prethreaded suture anchor attached to the driver bit of each inserter device, with the suture thread tautly attached to the inserter device and wound around the handle. The one or more inserter devices may be provided with one or more suture needles stored in the storage end, and the one or more suture needles can be prethreaded onto the suture thread or provided unthreaded. The kits can include comprise disposable inserter devices, wherein the entire inserter device may be thrown away after use. The kits may also comprise partially reusable inserter devices, wherein suture anchors, suture thread, and suture needles may be discarded after use while the inserter device may be retained and sterilized for further use by typical methods such as autoclaving, ultraviolet radiation, and the like.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A suture anchor inserter device comprising:
a substantially cylindrical handle positioned at a proximal end, the handle having an outer surface comprising at least one embedded groove circumscribing the handle, a planar surface adjacent to the at least one embedded groove, and a plurality of posts attached to the planar surface;
an elongate, substantially cylindrical shank extending from the handle towards a distal end, the shank having an outer surface, a lumen at least partially throughout, a window on the outer surface connected to the lumen, and a driver bit positioned at an open distal tip; and
a cap sized to fit over the planar surface of the handle and the at least one embedded groove.

2. The device of claim 1, wherein the plurality of posts are spaced to fit a suture needle wedged in-between.

3. The device of claim 1, wherein the at least one embedded groove forms a continuous channel circumscribing the handle.

4. The device of claim 1, wherein the handle further comprises a trough embedded in the outer surface, the trough extending from the at least one embedded groove to the shank.

5. The device of claim 1, wherein the shank comprises a peg extending from its outer surface, the peg being positioned near handle 16.

6. The device of claim 1, wherein the driver bit is selected from the group consisting of: a hex shape, a square shape, a triangle shape, a cross shape, a slot shape, and a Torx shape.

7. The device of claim 1, wherein the shank further comprises a trough embedded in its outer surface, the trough extending from the window to the handle.

8. The device of claim 1, wherein the cap is attached to the handle by a friction fit, a snap fit, or a threaded fitting.

9. The device of claim 1, wherein the planar surface is detachable from the handle along the at least one embedded groove.

10. The device of claim 1, further comprising a light source connected to a power supply.

11. The device of claim 10, wherein the light source is attached to the handle, the shaft, or both.

12. The device of claim 1, further comprising a drill bit positioned at the proximal end of the handle.

13. The device of claim 12, wherein the cap comprises a hole permitting the drill bit to extend through the cap.

14. A suture anchor inserter kit, comprising:
the suture anchor inserter device of claim 1;
at least one suture thread;
at least one suture anchor; and
at least one suture needle.

15. The kit of claim 14, wherein the at least one suture anchor is prethreaded with a length of suture thread.

16. The kit of claim 15, wherein the length of suture thread is prethreaded onto a suture needle.

17. The kit of claim 15, wherein the suture anchor is attached to the driver bit of the suture anchor inserter device, such that the suture thread is routed through the lumen of the shank, out of the window of the shank, and wound tautly around the at least one embedded groove of the handle.

18. The kit of claim 14, wherein the suture anchor inserter device is provided with at least one suture needled wedged in-between the plurality of posts.

19. The kit of claim 17, wherein the at least one suture needle is wedged in-between the plurality of posts and prethreaded with the length of suture thread wound tautly around the at least one embedded groove of the handle.

20. The kit of claim 19, wherein the cap is fitted over the planar surface of the handle and the at least one embedded groove and covers the length of suture thread wound around the at least one embedded groove and the at least one suture needle wedged in-between the plurality of posts.
